(19) United States
(12) Patent Application Publication
(10) Pub. No.: US 2017/0354357 A1
(43) Pub. Date: Dec. 14, 2017

(54) SYSTEMS, METHODS, AND DEVICES FOR MONITORING TISSUE ABLATION USING TISSUE AUTOFLUORESCENCE

(71) Applicant: Boston Scientific Scimed Inc., Maple Grove, MN (US)

(72) Inventors: Jacob I. Laughner, St. Paul, MN (US); Sarah R. Gutbrod, St. Paul, MN (US); Jason J. Hamann, Blaine, MN (US); Matthew S. Sulkin, New Brighton, MN (US); Mary M. Byron, Roseville, MN (US); Devon N. Arnholt, Shoreview, MN (US)

(21) Appl. No.: 15/619,410

(22) Filed: Jun. 9, 2017

Related U.S. Application Data
(60) Provisional application No. 62/348,901, filed on Jun. 11, 2016.

(51) Int. Cl. A61B 5/145; A61B 5/1459; A61B 5/02; A61B 5/00; A61B 18/14; A61B 18/00

(52) U.S. Cl. CPC A61B 5/14546; A61B 5/6852; A61B 18/1492; A61B 5/1459; A61B 5/0071; A61B 5/02; A61B 2018/00577; A61B 2018/00351

(57) ABSTRACT

A catheter system includes a catheter with an elongate catheter body, a catheter tip coupled to a distal end of the catheter body, and at least one light-emitting element configured to emit light to excite flavin adenine dinucleotide (FAD) molecules. The catheter system further including at least one light sensor configured to sense a light emitted by the excited FAD molecules.

### CROSS-REFERENCE TO RELATED APPLICATION

This application claims priority to Provisional Application No. 62/348,901, filed Jun. 11, 2016, which is herein incorporated by reference in its entirety.

### TECHNICAL FIELD

The present disclosure relates to analyzing anatomical structures within the body. More specifically, the present disclosure relates to systems, methods, and devices for monitoring and assessing tissue ablation using fluorescence.

### BACKGROUND

In ablation therapy, it is often necessary to determine various characteristics of body tissue at a target ablation site within the body. In interventional cardiac electrophysiology procedures, for example, it is often necessary for the physician to determine the condition of cardiac tissue at a target ablation site in or near the heart.

### SUMMARY

In Example 1, a catheter system includes a catheter with an elongate catheter body, a catheter tip coupled to a distal end of the catheter body, and at least one light-emitting element configured to emit light to excite flavin adenine dinucleotide (FAD) molecules. The catheter system further including at least one light sensor configured to sense a light emitted by the excited FAD molecules.

In Example 2, the catheter system of Example 1, wherein the at least one light-emitting element is a light-emitting diode.

In Example 3, the catheter system of Example 1, wherein the at least one light-emitting element is an optical fiber.

In Example 4, the catheter system of Example 3, wherein the optical fiber is communicatively coupled to a light source.

In Example 5, the catheter system of any of Examples 1-4, wherein the at least one light-emitting element is at least partially positioned within the catheter tip.

In Example 6, the catheter system of any of Examples 1-5, wherein the catheter tip includes at least one window or optically-transparent balloon.

In Example 7, the catheter system of any of Examples 1-6, wherein the at least one light sensor is at least partially positioned within the catheter tip.

In Example 8, the catheter system of any of Examples 1-7, further comprising control circuitry configured to assess lesion formation in response to receiving a signal indicative of an intensity of the sensed light emitted by the excited FAD molecules.

In Example 9, the catheter system of Example 8, wherein the control circuitry assesses lesion formation by determining the intensity of the sensed light is greater than a threshold.

In Example 10, the catheter system of Example 8, wherein the control circuitry assesses lesion formation by determining that a rate of change in intensity of the sensed light is greater than a threshold.

In Example 11, the catheter system of Example 8, wherein the control circuitry assesses lesion formation by determining that a difference in intensity of the sensed light is greater than a threshold.

In Example 12, a method for assessing tissue damage includes receiving an indication of intensity of FAD fluorescence of a section of tissue; and in response to the received indication of intensity, determining whether the section of tissue is damaged by an ablation catheter.

In Example 13, the method of Example 12, further comprising: determining, based on the received indication of intensity, whether an ablation catheter has contacted the section of tissue.

In Example 14, the method of any of Examples 12-13, wherein determining whether the section of tissue is damaged further comprises determining that the intensity is greater than a threshold.

In Example 15, the method of any of Examples 12-14, wherein determining whether the section of tissue is damaged further comprises determining that a rate of change of intensity is greater than a threshold.

In Example 16, a system includes a catheter with an elongate catheter body, a catheter tip coupled to a distal end of the catheter body, and at least one optical fiber partially positioned within the catheter tip and configured to emit light to excite flavin adenine dinucleotide (FAD) molecules. The system further includes at least one light sensor communicatively coupled to the at least one optical fiber and configured to sense a light emitted by the excited FAD molecules.

In Example 17, the system of Example 16, wherein the catheter further comprises at least one ablation electrode coupled to a catheter tip.

In Example 18, the system of any of Examples 16-17, wherein the catheter tip includes at least one window coupled to the at least one optical fiber.

In Example 19, the system of Example 18, wherein the at least one window is positioned on a distal end of the catheter tip.

In Example 20, the system of any of Examples 16-19, wherein the catheter further includes a plurality of optical fibers.

In Example 21, the system of any of Examples 16-20, wherein the catheter further includes a lumen extending within and along the catheter body, wherein the plurality of optical fibers are wound around the lumen.

In Example 22, the system of any of Examples 16-21, wherein at least a portion of the plurality of optical fibers are positioned radially around a circumferential wall of the catheter tip.

In Example 23, the system of any of Examples 16-22, wherein at least one of the plurality of optical fibers terminates at a distal end of the catheter tip.

In Example 24, the system of any of Examples 16-23, wherein at least one of the plurality of optical fibers terminates at a circumferential wall of the catheter tip.

In Example 25, the system of any of Examples 16-24, further comprising: control circuitry configured to assess lesion formation in response to receiving a signal indicative of an intensity of the sensed light emitted by the excited FAD molecules.

In Example 26, the system of any of Examples 16-25, wherein the control circuitry is configured to assess lesion formation by determining the intensity of the sensed light is greater than a threshold.

In Example 27, the system of any of Examples 16-25, wherein the control circuitry is configured to assess lesion formation by determining that a rate of change in intensity of the sensed light is greater than a threshold.

In Example 28, the system of any of Examples 16-25, wherein the control circuitry is configured to assess lesion formation by determining that a difference in intensity of the sensed light is greater than a threshold.

In Example 29, the system of any of Examples 16-28, wherein the at least one light sensor in one of a photodetector, spectrophotometer, camera, semiconductor, and photomultiplier tube.

In Example 30, a method for assessing tissue damage includes receiving an indication of intensity of flavin adenine dinucleotide (FAD) fluorescence near a section of tissue; and in response to the received indication of intensity, determining whether the section of tissue is damaged.

In Example 31, the method of Example 30, further comprising: receiving an indication of intensity of nicotinamide adenine dinucleotide hydrogen (NADH) fluorescence near a section of tissue; and in response to the received indication of NADH fluorescence intensity, determining whether the section of tissue is damaged.

In Example 32, the method of any of Examples 30-31, further comprising: exciting FAD molecules by directing light towards the section of tissue; and detecting FAD fluorescence intensity of the excited FAD molecules.

In Example 33, the method of any of Examples 30-32, wherein determining whether the section of tissue is damaged includes: determining the intensity of the sensed light is greater than a threshold.

In Example 34, the method of any of Examples 30-33, wherein determining whether the section of tissue is damaged includes: determining that a rate of change in intensity of the sensed light is greater than a threshold.

In Example 35, the method of any of Examples 30-34, wherein determining whether the section of tissue is damaged includes: determining that a difference in intensity of the sensed light is greater than a threshold.

In Example 36, a method includes receiving an indication of intensity of FAD fluorescence of a section of tissue. In response to the received indication of intensity, the method further includes determining whether an ablation catheter has contacted the section of tissue.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 shows a catheter system, in accordance with certain embodiments of the present disclosure.

FIG. 2 shows a schematic side view of a portion of a catheter, in accordance with certain embodiments of the present disclosure.

FIG. 3 shows a cut-away schematic side view of the catheter of FIG. 2.

FIG. 4 shows a schematic side view of a portion of a catheter, in accordance with certain embodiments of the present disclosure.

FIG. 5 shows a cut-away schematic side view of the catheter of FIG. 4.

FIG. 6 shows a cut-away schematic side view of a catheter, in accordance with certain embodiments of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the invention as defined by the appended claims.

### DETAILED DESCRIPTION

Various cardiac abnormalities can be attributed to improper electrical activity of cardiac tissue. Such improper electrical activity can include, but is not limited to, generation of electrical signals, conduction of electrical signals, and/or compression of the tissue in a manner that does not support efficient and/or effective cardiac function. For example, an area of cardiac tissue may become electrically active prematurely or otherwise out of synchrony during the cardiac cycle, causing the cardiac cells of the area and/or adjacent areas to contract out of rhythm. The result is an abnormal cardiac contraction that is not timed for optimal cardiac output. In some cases, an area of cardiac tissue may provide a faulty electrical pathway (e.g., a short circuit) that causes an arrhythmia, such as atrial fibrillation or supraventricular tachycardia. In some cases, inactive tissue (e.g., scar tissue) may be preferable to malfunctioning cardiac tissue.

Cardiac ablation is a procedure by which cardiac tissue is treated to inactivate the tissue. The tissue targeted for ablation may be associated with improper electrical activity, as described above. Cardiac ablation can lesion the tissue and prevent the tissue from improperly generating or conducting electrical signals. For example, a line, a circle, or other formation of ablated cardiac tissue can block the propagation of errant electrical signals. In some cases, cardiac ablation is intended to cause the death of cardiac tissue and to have scar tissue reform over the lesion, where the scar tissue is not associated with the improper electrical activity. Ablation therapies include radiofrequency ablation, cyroablation, microwave ablation, laser ablation, and surgical ablation, among others.

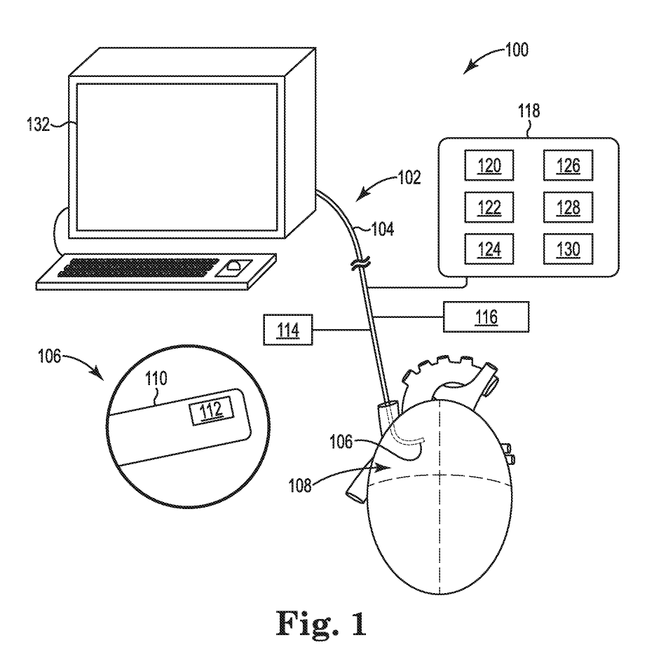

FIG. 1 shows a system 100 including a catheter 102 comprising an elongated catheter body 104 and a catheter tip 106, which is configured to be positioned within a heart 108. The catheter 102 includes an ablation electrode 110 coupled to the catheter tip 106. In operation, the ablation electrode 110 contacts targeted cardiac tissue to deliver ablative energy to the cardiac tissue, thus ablating the tissue to form a lesion, which can treat cardiac rhythm disturbances or abnormalities. The catheter 102 also includes at least one light-emitting or imaging element 112 such as an optical fiber (e.g., a light-emitting element) that transmits light to and from the catheter 102, an imaging sensor (e.g., an imaging element) that detects light, and/or a catheter-based light source (e.g., a light-emitting element) such as an light-emitting diode—each of which are discussed in more detail below.

When the element 112 is an optical fiber, the system 100 can include at least one sensor 114 that is coupled to the at least one optical fiber and that is configured to sense light transmitted by the at least one optical fiber. Such a configuration can also include a light source 116 (e.g., laser(s)) coupled to the at least one optical fiber to supply light to the at least one optical fiber, which transmits the supplied light through the catheter 102.

The system 100 also includes control circuitry 118 (including a memory 120, processor 122, measurement sub-unit 124, analyzer sub-unit 126, mapping sub-unit 128, and display controller 130) and a display 132. As will be explained in more detail below, the system 100 and its various components are configured to utilize fluorescence to assess and monitor tissue ablation (e.g., lesion formation).

In cardiac tissue, mitochondria assist in meeting energy demands of a beating heart. Nicotinamide adenine dinucleotide (NAD) and flavin adenine dinucleotide (FAD) are two elements of aerobic energy production within the mitochondria and have an intrinsic fluorescence. In addition to assisting with meeting energy demands, mitochondria assist with cell death mechanisms. In necrosis, the cell death mechanism associated with ablation, swelling and destruction of intracellular organelles leads to an increase in cell death signaling, among other things—culminating in a collapse of the mitochondrial membrane and oxidation of NADH and FADH₂. Oxidized FADH₂ (i.e., FAD) emits light when excited by light, and the intensity of the emitted light can be an indicator of the metabolic state of the tissue. The intensity of the emitted light thus can be an indicator of tissue health for real-time assessment and monitoring of tissue ablation (e.g., lesion formation). Certain embodiments of the present disclosure are accordingly directed to utilizing FAD and its fluorescence to assess and monitor lesion formation and therefore assist with tissue ablation.

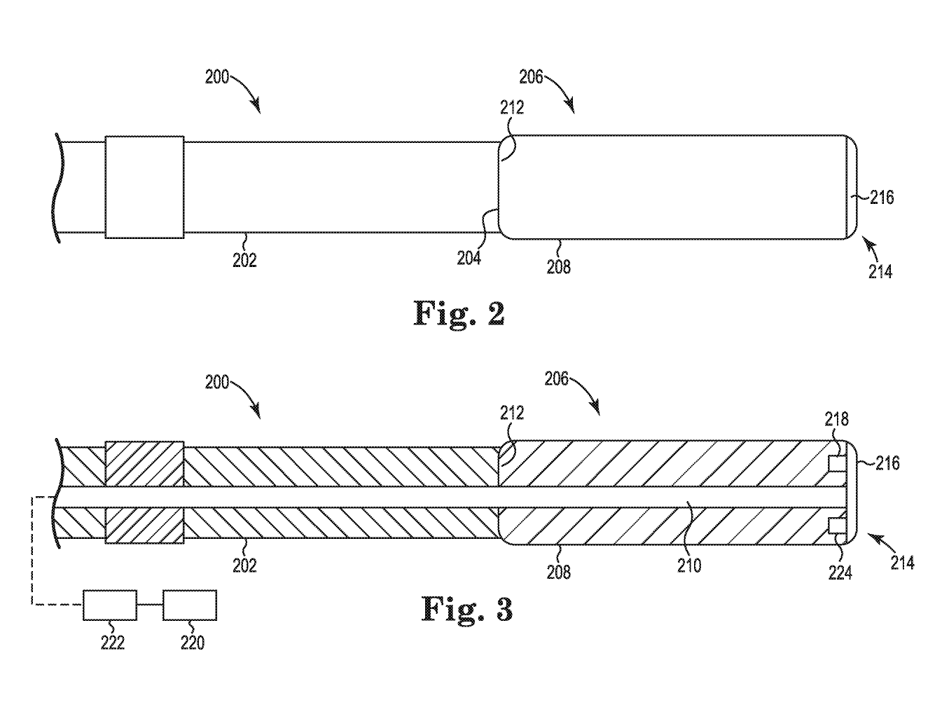

FIG. 2 is a schematic illustration of a side view of a portion of a catheter 200 that can be used in the system 100 of FIG. 1. FIG. 3 shows a cut-away schematic view of the catheter 200. The catheter 200 includes a catheter body portion 202 having a distal end 204 that is coupled to a catheter tip 206. The catheter tip 206 comprises an ablation electrode 208 that is configured to deliver ablative energy to cardiac tissue to form lesions along the tissue.

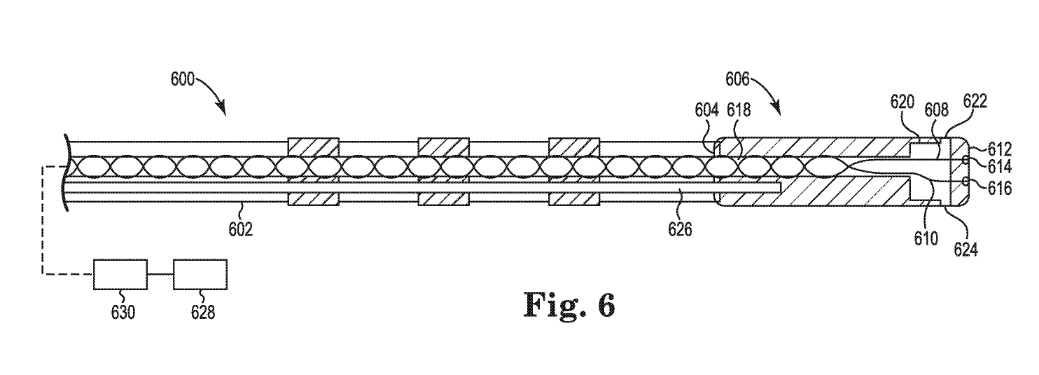

The catheter 200 includes a light-emitting element, which can be an optical fiber 210 and/or a catheter-based light source 218. In certain embodiments, the optical fiber 210 extends through the catheter tip 206 (e.g., from a proximal end 212 to a distal end 214 of the catheter tip 206) and is coupled to a window 216 positioned at the distal end 214 of the catheter tip 206. Although only one optical fiber is shown in FIG. 3, a plurality of optical fibers can be utilized, as shown in FIGS. 5-6. The optical fiber 210 can be braided together with other optical fibers and/or communication/electrical wires along the catheter 200 and/or wrapped around other components of the catheter 200 such as lumens. Although not shown in FIGS. 2-3, the catheter 200 can include various mapping and/or navigation sensors to assist with gathering physiological parameters and catheter positioning information and directing such information to the control circuitry 118 of the system 100.

The optical fiber 210 is configured to direct light (e.g., ultraviolet light) through the window 216. The window 216 can comprise glass, acrylics, silicone, polycarbonate (Lexan), plexiglass, fluorinated ethylene propylene (FEP), optically-clear epoxies, and the like. In certain embodiments, the optical fiber 210 is communicatively coupled to a light source (like the light source 116 shown in FIG. 1), which supplies light to the optical fiber 210. In certain embodiments, the catheter 200 includes the catheter-based light source 218 that is positioned within the catheter tip 206. The catheter-based light source 218 can be a light-emitting diode (LED) and the like.

The light-emitting element (e.g., optical fiber 210 or catheter-based light source 218) can be coupled to a steering mechanism (not shown), which can articulate either the optical fiber 210 or the catheter-based light source 218 in different directions such that the light emitted from the optical fiber 210 or the catheter-based light source 218 can be directed in a specific direction. For example, the optical fiber 210 or the catheter-based light source 218 can be rotated to direct light through different sections of the window 216 and at different directions. Although the window 216 is shown as being positioned at the distal end 214 of the catheter tip 206, the window 216 could cover a larger portion of the catheter tip 206 to provide a larger range of directions at which the optical fiber 210 or the catheter-based light source 218 can direct light. In some embodiments, the window 216 does not cover the entire distal end 214 of the catheter tip 206. In certain embodiments, the catheter 200 includes a balloon (e.g., optically-transparent balloon) in place of or in addition to the catheter tip 206. An optically-transparent balloon can provide a large range of directions at which the optical fiber 210 or the catheter-based light source 218 can direct light.

The light source (e.g., light source 116 in FIG. 1) supplies light to the optical fiber 210—or the catheter-based light source 218 emits light—at a desired wavelength or range of wavelengths (e.g., ultraviolet (UV) light). Specifically, the optical fiber 210 or the catheter-based light source 218 is configured to direct light within a patient at a wavelength such that FAD molecules are excited. As previously mentioned, when excited, FAD emits light and the intensity of the emitted light can be an indicator of the metabolic state of the issue and therefore an indicator of tissue health. When excited by approximately 405-500 nm UV light (with peak excitation occurring at approximately 460 nm), FAD emits light between approximately 500-600 nm (with peak emission at approximately 535 nm). As such, the optical fiber 210 or the catheter-based light source 218 can be configured to direct light at and/or between 405-500 nm.

In certain embodiments, the optical fiber 210 is communicatively coupled to at least one sensor 220 such that the optical fiber 210 transmits light (e.g., FAD fluorescence) to the at least one sensor 220, which is configured to sense the transmitted light (e.g., FAD fluorescence, optical data). For example, the sensor 220 can be but is not limited to a photodetector, spectrophotometer, camera (e.g., MEMS-based camera), semiconductor (e.g., CMOS), photomultiplier tube, among other imaging and light detection devices and/or circuitry. The sensor 220 can be coupled to one or more filters 222 such as a filter designed to filter out wavelengths outside the emission range of FAD and/or NADH.

In certain embodiments, the catheter 200 includes a sensor 224 that is positioned on or within the catheter tip 206. The sensor 224 can be photodetector, spectrophotometer, camera (e.g., MEMS-based camera), semiconductor (e.g., CMOS), photomultiplier tube, among other imaging and light detection devices and/or circuitry sized to fit onto or within the catheter tip 206. Embodiments utilizing the catheter-based light source 218 and the sensor 224 may not need to use the optical fiber 210, the light source 116, and/or the sensor 220.

During an ablation procedure, the catheter 200 is positioned adjacent tissue that is to be ablated. If the optical fiber 210 or the catheter-based light source 218 were to direct light towards the tissue before ablation, the sensor 220 or 224 would sense little to no intensity of FAD fluorescence because, before being ablated, little to no FAD molecules would be produced from FADH₂ oxidation. However, once the ablation electrode 208 is energized and moved into contact with the tissue, FADH₂ begin to oxidize into FAD molecules. When light from the optical fiber 210 or the catheter-based light source 218 is directed towards the tissue, the FAD molecules become excited and emit light. More specifically, when the optical fiber 210 or the catheter-based light source 218 directs light with a wavelength of 405-500 nm, FAD emits light (e.g., FAD fluorescence) between approximately 500-600 nm, which is detected by the at least one sensor 220 or 224.

The intensity of the emitted light (e.g., FAD fluorescence intensity) can be measured, analyzed, and/or used to assess and monitor ablation of the tissue. For example, various thresholds can be predetermined or dynamically varied and used to determine when a catheter's ablation electrode has contacted tissue and/or ablation of the tissue has started, is occurring, is successful, and/or has ended. The thresholds can be based on parameters such as a levels of intensity, rates of increase in intensity (e.g., slopes), timing of events involving intensity and/or in combination with other physiological parameters, and levels of intensity changes (e.g., deltas), etc. In certain embodiments, an increase in the level of intensity can indicate that tissue ablation has started because FAD fluorescence intensity generally increases once ablation has started. In certain embodiments, certain rates of increase in the level of intensity can indicate that tissue ablation has started. In certain embodiments, an increase and subsequent plateau in a level of intensity can indicate that the target tissue is fully ablated. For example, a plateau of the level of intensity for a certain amount of time (e.g., 15, 30, 45, or 60 seconds) can indicate that the target tissue is fully ablated. In certain embodiments, FAD fluorescence intensity can be synchronized with other physiological parameters. For example, levels of FAD fluorescence intensity can be synchronized, and plotted against, ECG signals and local electrograms, among other physiological parameters.

Optical data, such as fluorescence intensity, can be used to normalize FAD fluorescence intensity by using a broader range of light reflected from the excitation source. For example, when the optical fiber 210 or the catheter-based light source 218 emits light at the peak excitation wavelength (e.g., 460 nm), the measured FAD fluorescence intensity resulting from tissue ablation can be divided by the measured fluorescence intensity outside the FAD wavelength band (e.g., outside 500-600 nm). The optical data can also be used to generate “smart tags” within an electro-anatomic mapping system. For example, during or after creating a map of portions of a heart (e.g., an electro-anatomic shell), a tag can be automatically placed on the map at areas where FAD fluorescence intensity (or another fluorescence-related parameter) is determined to be greater than a threshold, such as the various thresholds discussed above. In certain embodiments, the tag can be automatically created by setting a threshold on cumulative FAD fluorescence over time (e.g., fluorescence-time integral). The tag may be visually encoded with the maximum FAD fluorescence intensity or cumulative FAD fluorescence over time.

Although the description above primarily discusses using FAD fluorescence intensity, parameters other than or in addition to intensity can be used. These other parameters can include parameters that characterize how fluorescence changes over time. For example, lifetime characteristics of fluorescence (e.g., how quickly fluorescence changes, how does fluorescence recover after photo-bleaching) can be measured and controlled by timing of excitation light (e.g., pulsing excitation light) and timing of light collection.

Assessment and monitoring of tissue ablation can be carried out using various components of the system 100. The control circuitry 118 includes memory 120, processor 122, measurement sub-unit 124, analyzer sub-unit 126, mapping sub-unit 128, and display controller 130. The control circuitry 118 and its components are configured to carry out the various functions of the system. In operation, FAD fluorescence detected by the at least one sensor 220 or 224 is communicated to the measurement sub-unit to determine the intensity of the FAD fluorescence. Once the FAD fluorescence intensity is determined, the analyzer sub-unit 126 compares the intensity to certain predetermined or dynamic thresholds (as mentioned above) to assess whether the ablation electrode 208 has contacted issue and/or whether tissue ablation has started, is occurring, is successful, and/or has ended. The mapping sub-unit 128 receives mapping/positioning signals from mapping and/or navigation sensors coupled to the catheter 200 and determines physiological mapping and catheter position information. The display controller 130 outputs the results of the various sub-units to the display 132. For example, the display controller 130 can combine mapping, positioning, and FAD fluorescence intensity information and output such information to the display 132, which can indicate that certain portions of a targeted ablation site are not fully ablated. Such information can be gathered and displayed in real-time to assist with monitoring and assessing lesion formation. In some embodiments, the display controller 130 synchronizes, in time, signals associated with the determined FAD fluorescence intensity and signals associated with other physiological parameters such that the display 132 displays the synchronized signals.

The control circuitry 118 can include a computer-readable recording medium or “memory” 120 for storing processor-executable instructions, data structures and other information. The memory 120 may comprise a non-volatile memory, such as read-only memory (ROM) and/or flash memory, and a random-access memory (RAM), such as dynamic random access memory (DRAM), or synchronous dynamic random access memory (SDRAM). In some embodiments, the memory 120 may store processor-executable instructions that, when executed by a processor 122, perform routines for carrying out the functions related to assessing and monitoring tissue ablation.

In addition to the memory 120, the control circuitry 118 may include other computer-readable media storing program modules, data structures, and other data described herein for assessing and monitoring tissue ablation. It will be appreciated by those skilled in the art that computer-readable media can be any available media that may be accessed by the control circuitry 118 or other computing system for the non-transitory storage of information. Computer-readable media includes volatile and non-volatile, removable and non-removable recording media implemented in any method or technology, including, but not limited to, RAM, ROM, erasable programmable ROM (EPROM), electrically-erasable programmable ROM (EEPROM), FLASH memory or other solid-state memory technology, compact disc ROM (CD-ROM), digital versatile disk (DVD), BLU-RAY or other optical storage, magnetic tape, magnetic disk storage or other magnetic storage devices and the like.

It will be appreciated that the structure and/or functionality of the control circuitry 118 may be different than that illustrated in FIG. 1 and described herein. For example, the processor 122, measurement sub-unit 124, analyzer sub-unit 126, mapping sub-unit 128, and display controller 130, and other components of the control circuitry 118 may be integrated within a common integrated circuit package or distributed among multiple integrated circuit packages. It will be further appreciated that the control circuitry 118 may not include all of the components shown in the FIG. 1, may include other components that are not explicitly shown in FIG. 1, or may utilize an architecture different than that shown in FIG. 1.

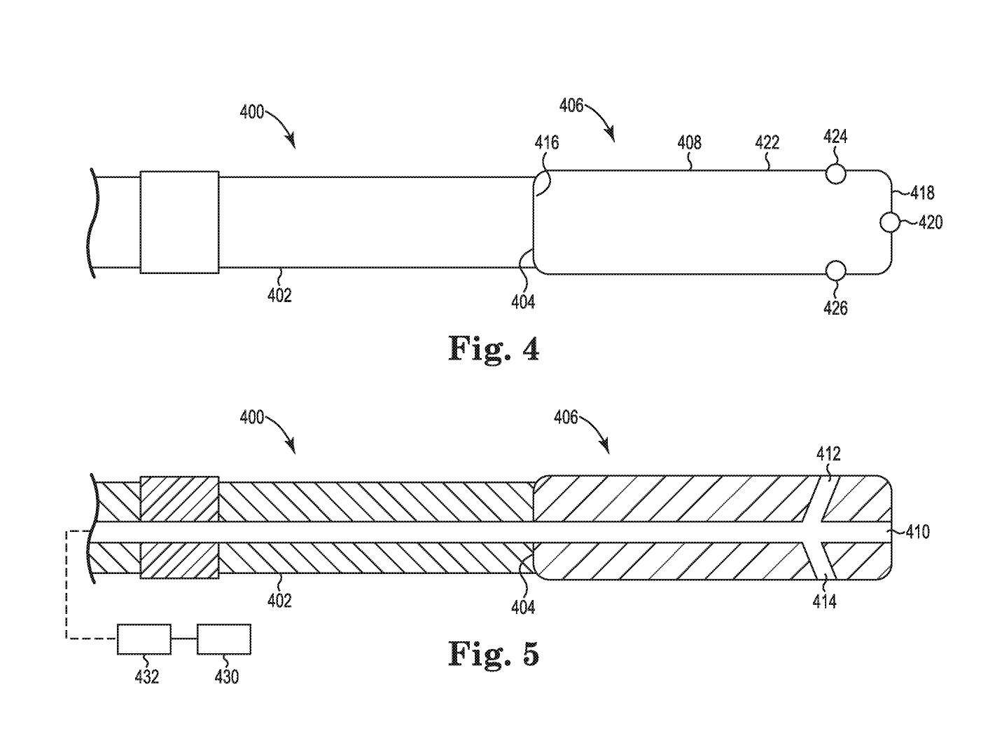

FIG. 4 shows a side view of a portion of a catheter 400 that can be used within the system 100 of FIG. 1. FIG. 5 shows a cut-away view of the catheter 400. The catheter 400 includes a catheter body portion 402 having a distal end 404 that is coupled to a catheter tip 406. The catheter tip 406 comprises an ablation electrode 408 that is configured to deliver ablative energy to cardiac tissue to form lesions along the tissue.

The catheter 400 includes multiple optical fibers 410, 412, and 414. Alternatively, or in addition, the catheter 400 could include one or more or the catheter-based light sources such as the catheter-based light source 218 shown in FIG. 3. For simplicity, FIGS. 4 and 5 show three optical fibers but the catheter 400 can include more or fewer optical fibers. One optical fiber 410 extends through the catheter tip 406 (e.g., from a proximal end 416 to a distal end 418 of the catheter tip 406) and is coupled to a window 420 positioned at the distal end 418 of the catheter tip 406. The other optical fibers 412, 414 are radially distributed and positioned along a circumferential wall 422 of the catheter tip 406 and are each coupled to a window 424, 426 respectively also positioned along the circumferential wall 422. In some embodiments, multiple optical fibers are positioned at the distal end 418 of the catheter tip 406. In other embodiments, all the optical fibers are positioned along the circumferential wall 422 of the catheter tip 406. In some embodiments, one optical fiber is communicatively coupled to multiple windows. With multiple fibers and/or multiple windows, the catheter 400 can direct light in multiple directions, which can help prevent signal loss in the event there is interference between tissue and light from one or more of the optical fibers.

The optical fibers are communicatively coupled to a light source (like the light source 116 shown in FIG. 1), which supplies light to the optical fibers. The optical fibers can be communicatively coupled to at least one sensor 430 such that the optical fibers transmit light to the at least one sensor 430, which is configured to sense the transmitted light (e.g., optical data). When a single light sensor is used, the sensed light from the single light sensor can multiplexed. In certain embodiments with more than one sensor, signals from each sensor can be multiplexed and directed to the control circuitry 118 for analysis. The at least one sensor 430 can be coupled to one or more filters 432 such as a filter designed to filter out wavelengths outside the emission range of FAD and/or NADH.

FIG. 6 shows a cut-away view of a portion of a catheter 600 that can be used within the system 100 of FIG. 1. The catheter 600 includes a catheter body portion 602 having a distal end 604 that is coupled to a catheter tip 606. The catheter 600 is shown as having two optical fibers 608, 610 although more or fewer optical fibers can be used. Both optical fibers 608, 610 extend through the catheter tip 606 to a distal end 612 and are coupled to window 614, 616 positioned at the distal end 612 of the catheter tip 606—although one or both of the windows could be positioned around a circumferential wall of the catheter tip 606. The optical fibers 608, 610 are communicatively coupled to one or more light sources (like the light source 116 shown in FIG. 1), which supplies light to the optical fibers.

FIG. 6 shows the optical fibers 608, 610 braided or wrapped around a lumen 618. Such configuration can provide strain relief to the optical fibers 608, 610 when the catheter 600 is articulated (e.g., bent). In certain embodiments, the lumen 618 can extending along and within at least a portion of the catheter body portion 602 and the catheter tip 606. In an irrigated catheter, the lumen 618 directs fluid (e.g., coolant) through the catheter body portion 602 and to the catheter tip 606 to cool the catheter tip 606 and/or tissue. For example, the lumen 618 can direct fluid to an interior cavity 620 of the catheter tip 606 and then to openings 622, 624 positioned around the catheter tip 606 through which fluid exits the catheter tip 606. The catheter 600 further includes a navigation sensor 626, which assists with indicating a location of the catheter tip 606 when positioned within a patient.

In certain embodiments, one of the optical fibers 608 is configured to emit light at a wavelength that excites FAD molecules, as discussed in detail above. The other fiber 610 is configured to emit UV light at a wavelength that excites NADH molecules. For example, the optical fiber 610 can emit UV light at a wavelength at or between 300-400 nm, which excites NADH molecules. When excited, NADH molecules emit light between 435-485 nm. But, unlike FAD, intensity of NADH fluorescence decreases as tissue is ablated. For example, a high NADH fluorescence intensity corresponds to unablated tissue. Thus, it can be challenging to determine whether a decrease in NADH fluorescence is due to ablation (e.g., increased lesion formation) or due to a catheter being moved away from tissue intended to be ablated. However, exciting FAD in addition to NADH and detecting their respective fluorescence can provide information to offset the certain disadvantages mentioned and associated with using NADH as an indicator of tissue ablation.

The optical fibers 608,610 can be communicatively coupled to at least one sensor 628 such that one or more of the optical fibers transmit light to the at least one sensor 628, which is configured to sense the transmitted light (e.g., optical data). When a single light sensor is used, the sensed light from the single light sensor can multiplexed. In certain embodiments with more than one sensor, signals from each sensor can be multiplexed and directed to the control circuitry 118 for analysis. The at least one sensor 628 can be coupled to one or more filters 630 such as a filter designed to filter out certain wavelengths.

Although certain embodiments of the disclosure feature an ablation electrode, monitoring and assessment of tissue ablation via optical fibers, sensors, control circuitry, etc. could be used with devices such as accessary and/or therapeutic catheters that do not include an ablation electrode. For example, an accessory or therapeutic catheter could monitor FAD fluorescence (and therefore tissue ablation) by positioning the catheter near the ablation site during ablation. In certain embodiments, the catheter includes a balloon (e.g., optically-transparent balloon) that facilitates transmission of light between the catheter and tissue. Moreover, features of the present disclosure are applicable to all ranges of ablation targets (e.g., atrial fibrillation, ventricular tachycardia) and ablation techniques (e.g., cyroablation, microwave ablation). For example, in certain embodiments, FAD fluorescence is enhanced at colder temperatures like those used with cryoablation.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A system comprising:
a catheter including:
an elongate catheter body,
a catheter tip coupled to a distal end of the catheter body, and
at least one optical fiber partially positioned within the catheter tip and configured to emit light to excite flavin adenine dinucleotide (FAD) molecules; and
at least one light sensor communicatively coupled to the at least one optical fiber and configured to sense a light emitted by the excited FAD molecules.

2. The system of claim 1, wherein the catheter further comprises at least one ablation electrode coupled to a catheter tip.

3. The system of claim 1, wherein the catheter tip includes at least one window coupled to the at least one optical fiber.

4. The system of claim 3, wherein the at least one window is positioned on a distal end of the catheter tip.

5. The system of claim 1, wherein the catheter further includes a plurality of optical fibers.

6. The system of claim 5, wherein the catheter further includes a lumen extending within and along the catheter body, wherein the plurality of optical fibers are wound around the lumen.

7. The system of claim 5, wherein at least a portion of the plurality of optical fibers are positioned radially around a circumferential wall of the catheter tip.

8. The system of claim 5, wherein at least one of the plurality of optical fibers terminates at a distal end of the catheter tip.

9. The system of claim 8, wherein at least one of the plurality of optical fibers terminates at a circumferential wall of the catheter tip.

10. The system of claim 1, further comprising:
control circuitry configured to assess lesion formation in response to receiving a signal indicative of an intensity of the sensed light emitted by the excited FAD molecules.

11. The system of claim 10, wherein the control circuitry is configured to assess lesion formation by determining the intensity of the sensed light is greater than a threshold.

12. The system of claim 10, wherein the control circuitry is configured to assess lesion formation by determining that a rate of change in intensity of the sensed light is greater than a threshold.

13. The system of claim 10, wherein the control circuitry is configured to assess lesion formation by determining that a difference in intensity of the sensed light is greater than a threshold.

14. The system of claim 1, wherein the at least one light sensor in one of a photodetector, spectrophotometer, camera, semiconductor, and photomultiplier tube.

15. A method for assessing tissue damage, the method comprising:
receiving an indication of intensity of flavin adenine dinucleotide (FAD) fluorescence near a section of tissue; and
in response to the received indication of intensity, determining whether the section of tissue is damaged.

16. The method of claim 15, further comprising:
receiving an indication of intensity of nicotinamide adenine dinucleotide hydrogen (NADH) fluorescence near a section of tissue; and
in response to the received indication of NADH fluorescence intensity, determining whether the section of tissue is damaged.

17. The method of claim 15, further comprising:
exciting FAD molecules by directing light towards the section of tissue; and
detecting FAD fluorescence intensity of the excited FAD molecules.

18. The method of claim 17, wherein determining whether the section of tissue is damaged includes: determining the intensity of the sensed light is greater than a threshold.

19. The method of claim 17, wherein determining whether the section of tissue is damaged includes: determining that a rate of change in intensity of the sensed light is greater than a threshold.

20. The method of claim 17, wherein determining whether the section of tissue is damaged includes: determining that a difference in intensity of the sensed light is greater than a threshold.
